(19) United States
(12) Patent Application Publication
(10) Pub. No.: US 2010/0191120 A1
(43) Pub. Date: Jul. 29, 2010

(54) APPARATUS AND METHOD FOR CONTROLLING AN ULTRASOUND SYSTEM BASED ON CONTACT WITH AN ULTRASOUND PROBE

(75) Inventors: Thomas Andrew Kraus, Waukesha, WI (US); Snehal C. Shah, Milwaukee, WI (US); Steven Charles Miller, Phoenix, AZ (US)

Correspondence Address: DEAN D. SMALL;THE SMALL PATENT LAW GROUP LLP, 225 S. MERAMEC, STE. 725T, ST. LOUIS, MO, 63105, US

(73) Assignee: GENERAL ELECTRIC COMPANY, Schenectady, NY (US)

(21) Appl. No.: 12/361,032

(22) Filed: Jan. 28, 2009

(51) Int. Cl. A61B 8/00

(52) U.S. Cl. 600/459

(57) ABSTRACT

An ultrasound probe comprises a probe housing that has an inner surface and an outer surface. An array of transducer elements are within the probe housing. At least one sensor is formed between the inner and outer surfaces of the probe housing. The at least one sensor is configured to detect at least one parameter associated with an object in contact with the outer surface proximate the at least one sensor.

### BACKGROUND OF THE INVENTION

This invention relates generally to ultrasound and more particularly to ultrasound probes.

Ultrasound exams often require the user to make many inputs and selections during the extent of the exam. The user makes selections through the ultrasound systems user interface, such as to input patient data, activate a probe, select and step through protocol(s), and to initiate other actions or adjustments to the system or probe, such as to change the scanning mode or a parameter of the probe. It can be time consuming for the user to locate and activate the appropriate selections on the keyboard or other user interface associated with the ultrasound system, and the user has to keep one hand free for making the selections.

To eliminate some of the user inputs, some conventional systems provide a mechanical switch that senses when the probe is removed from the probe holder, and thus activates and deactivates the probe based on the state of the switch. Also, some mechanical switches that may be used to activate one or more functions have been added to the probe or to devices that attach to the probe. However, mechanical switches can be easily damaged or wear out from use.

Therefore, there is a need to reduce user movement and to make the workflow more automatic while using the ultrasound system.

### BRIEF DESCRIPTION OF THE INVENTION

In one embodiment, an ultrasound probe comprises a probe housing that has an inner surface and an outer surface. An array of transducer elements are within the probe housing. At least one sensor is formed between the inner and outer surfaces of the probe housing. The at least one sensor is configured to detect at least one parameter associated with an object in contact with the outer surface proximate the at least one sensor.

In another embodiment, an ultrasound system comprises an ultrasound probe and a processor module. The ultrasound probe has a probe housing that has an inner surface and an outer surface. An array of transducer elements are within the probe housing, and at least one sensor is formed between the inner and outer surfaces of the probe housing. The at least one sensor is configured to detect a level of at least one parameter associated with an object in contact with the outer surface proximate the at least one sensor. The processor module is electrically coupled to the ultrasound probe, and is configured to initiate an action based on a relationship of the level of the at least one parameter to predetermined criteria.

In yet another embodiment, a method for controlling an ultrasound system based on capacitance changes detected proximate to an outer surface of an ultrasound probe comprises detecting with at least one capacitive sensor a level of capacitance on an outer surface of an ultrasound probe. The level of capacitance is compared to a capacitance criteria with a processor module, and an action is initiated with the processor module when the level of capacitance satisfies the capacitance criteria.

### BRIEF DESCRIPTION OF THE DRAWINGS

FIG. 1 illustrates an ultrasound system formed in accordance with an embodiment of the present invention.

FIG. 2 illustrates an exemplary cross-sectional view of a touch sensitive probe that has capacitive sensing incorporated within the housing of the probe in accordance with an embodiment of the present invention.

FIG. 3 illustrates another exemplary cross-sectional view of the touch sensitive probe that has capacitive sensing incorporated within the housing of the probe in accordance with an embodiment of the present invention.

FIG. 4 illustrates a plurality of capacitive sensors that are formed within a capacitive sensing layer of the touch sensitive probe in accordance with an embodiment of the present invention.

FIG. 5 illustrates capacitive sensing incorporated within an area of the touch sensitive probe in accordance with an embodiment of the present invention.

FIG. 6 illustrates virtual buttons that are associated with one or more capacitive sensors and formed within an area of the touch sensitive probe in accordance with an embodiment of the present invention.

FIG. 7 illustrates a method for using the touch sensitive probe that has at least one capacitive sensor integrated into the housing in accordance with an embodiment of the present invention.

FIG. 8 illustrates a 3D-capable miniaturized ultrasound system formed in accordance with an embodiment of the present invention.

FIG. 9 illustrates a mobile ultrasound imaging system formed in accordance with an embodiment of the present invention.

FIG. 10 illustrates a hand carried or pocket-sized ultrasound imaging system formed in accordance with an embodiment of the present invention.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

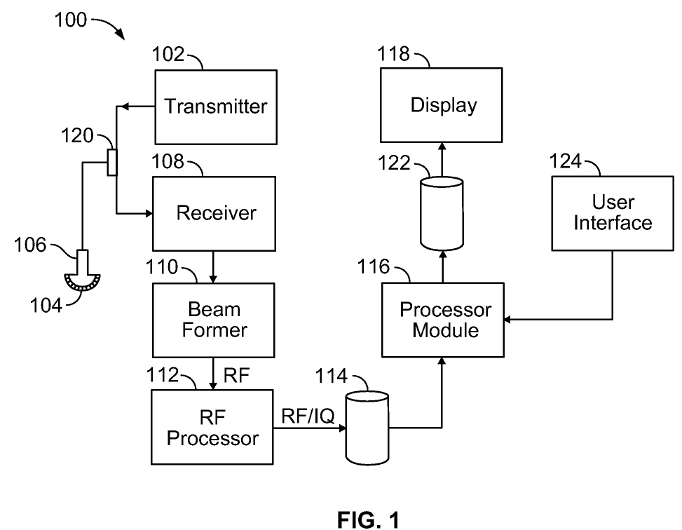

FIG. 1 illustrates an ultrasound system 100 including a transmitter 102 that drives an array of elements 104 (e.g., piezoelectric elements) within a probe 106 to emit pulsed ultrasonic signals into a body. The elements 104 may be arranged, for example, in one or two dimensions. A variety of geometries may be used. The system 100 may have a probe port 120 for receiving the probe 106 or the probe 106 may be hardwired to the system 100.

The ultrasonic signals are back-scattered from structures in the body, like fatty tissue or muscular tissue, to produce echoes that return to the elements 104. The echoes are received by a receiver 108. The received echoes are passed through a beamformer 110 that performs beamforming and outputs a radiofrequency (RF) signal. The RF signal then passes through an RF processor 112. Alternatively, the RF processor 112 may include a complex demodulator (not shown) that demodulates the RF signal to form in-phase and quadrature (IQ) data pairs representative of the echo signals. The RF or IQ signal data may then be routed directly to a memory 114 for storage.

The ultrasound system 100 also includes a processor module 116 to process the acquired ultrasound information (e.g., RF signal data or IQ data pairs) and prepare frames of ultrasound information for display on display 118. The processor module 116 is adapted to perform one or more processing operations according to a plurality of selectable ultrasound modalities on the acquired ultrasound information. Acquired ultrasound information may be processed and displayed in real-time during a scanning session as the echo signals are received. Additionally or alternatively, the ultrasound information may be stored temporarily in memory 114 or memory 122 during a scanning session and then processed and displayed in an off-line operation.

A user interface 124 may be used to input data to the system 100, adjust settings, and control the operation of the processor module 116. The user interface 124 may have a keyboard, trackball and/or mouse, and a number of knobs, switches or other input devices such as a touchscreen. The display 118 includes one or more monitors that present patient information, including diagnostic ultrasound images to the user for diagnosis and analysis. One or both of memory 114 and memory 122 may store two-dimensional (2D) and/or three-dimensional (3D) datasets of the ultrasound data, where such datasets are accessed to present 2D and/or 3D images. Multiple consecutive 3D datasets may also be acquired and stored over time, such as to provide real-time 3D or four-dimensional (4D) display. The images may be modified and the display settings of the display 118 also manually adjusted using the user interface 124.

Touch sensing technology (not shown in FIG. 1), such as capacitive sense technology, may be integrated or incorporated into the casing or housing of the probe 106 so that the processor module 116 of the system 100 may change or alter the status or state of the probe 106 and/or system 100 based on a user's proximity and/or contact with the housing. In other embodiments, other types of non-mechanical sensors may be used to detect a user's contact with the housing, such as resistance sensors, piezoelectric elements that may detect a level of pressure, inductive sensors, or any other sensor that causes a measurable change in one or more parameters (e.g. capacitance, inductance, resistance, and the like) in response to proximity and/or contact of the user with the housing. In some embodiments the parameter may be an electrical parameter. In yet another embodiment, a combination of different types of sensors may be used. A technical effect of at least one embodiment is that touch sensing technology, such as capacitive sense technology, may be used to discriminate between a user's touch (e.g. human or organic) and touch from other objects, such as non-organic objects like a table, probe holder, and the like. Capacitive sense technology may also detect capacitance changes that result from pressure changes. Therefore, at least one embodiment discussed herein provides method and apparatus for controlling operations of the probe 106 and the ultrasound system 100 based on the detection of the user's touch on the surface of the probe 106.

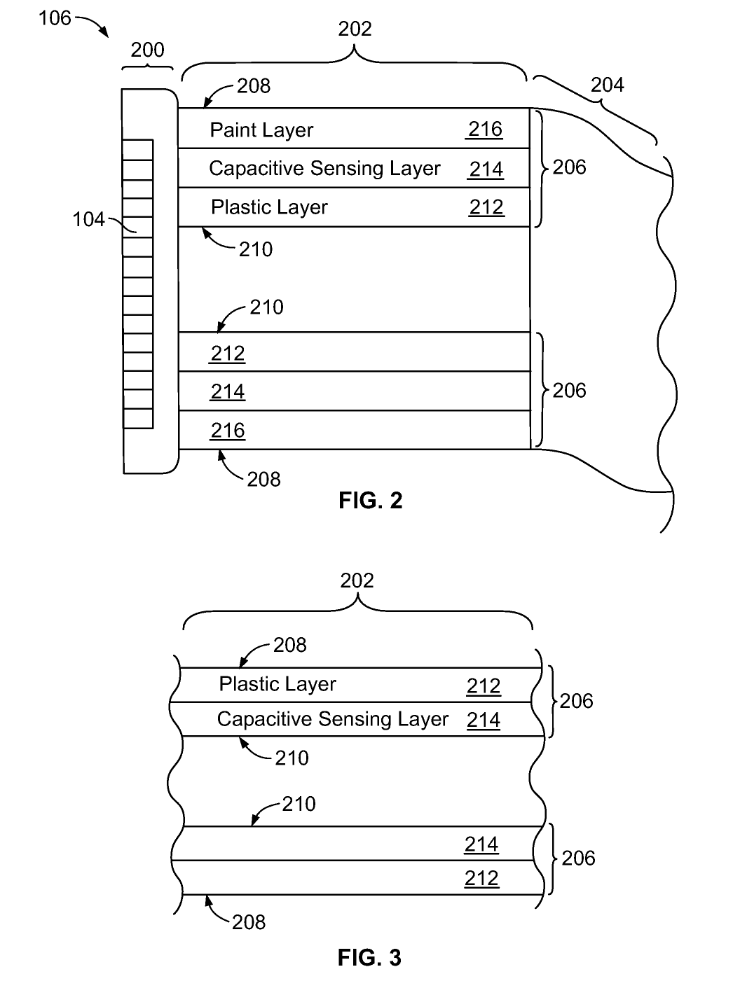

FIG. 2 illustrates an exemplary cross-sectional view of the touch sensitive probe 106. The probe 106 may be generally divided into three portions, namely, a scan head 200, a handle 202 and a cable 204. The transducer elements 104 are located in the scan head 200. The handle 202 has electronics and the like there-within (not shown) for selecting elements 104, conveying signals between the elements 104 and the cable 204 and/or processing signals. Wires, such as coaxial wires or cables (not shown) within the cable 204 convey signals to and from the probe 106 and the probe port 120.

A probe housing 206 having an outer surface 208 and an inner surface 210 encases the probe 106, preventing contaminants such as liquid and dust from interfering with the elements 104, the electronics, and wires within the probe 106. The probe housing 206 may be formed of one or more layers of material. In the embodiment shown in FIG. 2, a plastic layer 212 is formed nearest the inner surface 210. The layer 212 may be formed of material(s) other than plastic, such as a composite, rubber, silicon or other materials or combinations of materials. A capacitive sensing layer 214 is formed next to the plastic layer 212, and a paint layer 216 is formed nearest the outer surface 208. Therefore, the capacitive sensing layer 214 is formed between the outer and inner surfaces 208 and 210 of the probe housing 206. Although capacitive sensing technology is illustrated, it should be understood that in other embodiments, the capacitive sensing layer 214 may be replaced with other non-mechanical touch sensing technologies, a combination of non-mechanical touch sensing technologies, or a combination of non-mechanical and mechanical touch sensing technologies. For example, a resistive layer or an inductive layer may be used, or capacitive sensors may be formed within the same layer as resistive sensors. Other combinations are possible and are thus not restricted to the examples discussed herein.

In the embodiment shown in FIG. 3, the housing 206 does not have a paint layer. Instead, the plastic layer 212 is formed nearest the outer surface 208, and the capacitive sensing layer 214 is formed nearest the inner surface 210. By way of example, the plastic layer 212 may be colored, imprinted, or otherwise provided with the desired color, graphics and the like, such that an outer layer of paint is not needed. It should be understood that other layers (not shown) may be incorporated within the housing 206. In one embodiment, when the plastic layer 212 is positioned as shown in FIG. 3, the thickness of the plastic layer 212 may be determined based on the capability of the capacitive sensing layer 214, such as by limiting the thickness of the plastic layer 212 to five millimeters or less. Other thicknesses may be used based on at least the sensitivity of the capacitive sensing layer 214. In another embodiment, the capacitive sensing layer 214 may be integrated with or into the plastic layer 212, forming a single layer that may or may not have an associated paint layer or other layer positioned along either of the outer surface 208 or the inner surface 210.

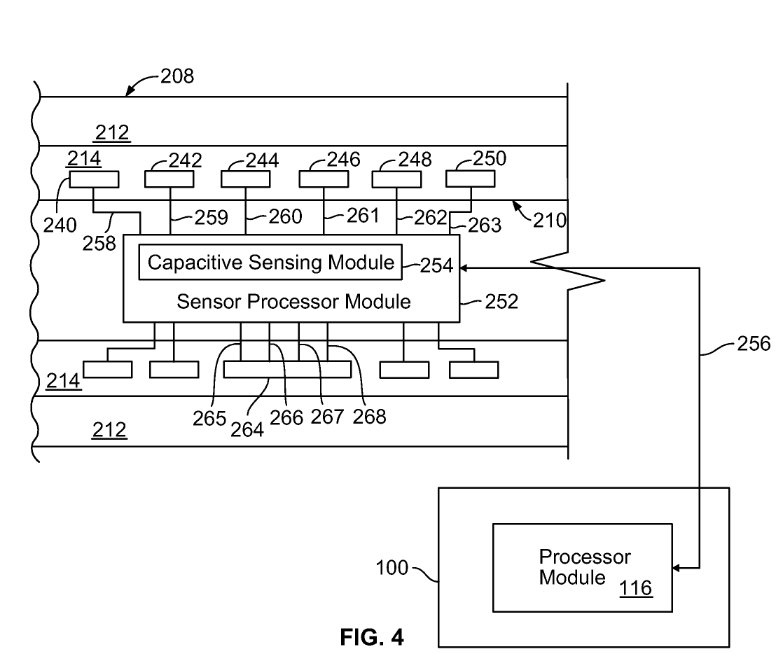

FIG. 4 illustrates a plurality of capacitive sensors 240, 242, 244, 246, 248 and 250 that are formed within the capacitive sensing layer 214. In another embodiment, the capacitive sensors 240-250 may be incorporated within the plastic layer 212. It should be understood that the number of capacitive sensors 240-250 illustrated is exemplary only, and that more or less capacitive sensors may be used. Also, the sensors 240-250 may be the same size or different sizes. Each of the capacitive sensors 240-250 senses a level of capacitance on the outer surface 208 proximate to the sensor. As discussed previously, sensors that sense other parameters on or near the outer surface 208, such as resistance, inductance, pressure or voltage, may be used to form a sensing layer, and may in some embodiments be used in combination with one or more of the capacitive sensors 240-250.

Regardless of how the capacitive sensors 240-250 or capacitive sensing layer 214 are incorporated within the housing 206 of the probe 106, the probe 106 is sealed from outer contaminants and thus the probe 106 may be cleaned, disinfected, sterilized and the like without harming the capacitive sensors 240-250 or capacitive sensing layer 214. Also, the capacitive sensors 240-250 have no moving parts and thus are not subject to mechanical fatigue and failure.

In one embodiment, each of the capacitive sensors 240-250 may be formed of a pair of adjacent electrodes or capacitors. One side of each of the capacitors may be grounded and the sensor 240-250 has an associated level of capacitance to ground when a conductive object is not present. When a conductive object is within a predetermined range of the sensor 240-250, such as when the conductive object is in contact with the outer surface 208, an electrical connection is made between the conductive object and the sensor 240-250 and the level of capacitance to ground increases.

A capacitive sensing module 254 within a sensor processor module 252 may be housed within the probe 106 and may monitor the level of capacitance of each of the sensors 240-250, such as through leads 258, 259, 260, 261, 262 and 263, respectively. For example, the signal from each of the sensors 240-250 may be a low level analog signal. Although not shown, an amplifier may be used to increase the level of the signal, such as to allow easier detection and comparison of the signal to ranges and thresholds. When the capacitance increases above a predetermined threshold or is within a predetermined range, the sensor processor module 252 may determine that the outer surface 208 proximate the sensor 240-250 has been touched by the user. In one embodiment, the capacitive sensing module 254 may provide a discrete output associated with one or more of the sensors 240-250, indicating that the sensor has or has not touched been by the user. Alternatively, outputs from the sensors 240-250 may be sensed by other circuitry (not shown), such as within the processor module 116 or elsewhere within the system 100. Therefore, it should be understood that other processors and circuitry may be used to sense the level of capacitance or otherwise determine that the sensor 240-250 has experienced a change in capacitance. In another embodiment, one or more sensor 264 that is configured to cover an area of the probe surface may be connected to the sensor processor module 252 through more than one lead 265, 266, 267 and 268. The level of capacitance on the leads 265-268 may be used to determine the presence of a touch as well as coordinate or X, Y location information of the touch within the area of the sensor 264.

The sensor processor module 252 may be electrically connected to the processor module 116 within the system 100 via coaxial wires 256 or other cables within the probe cable 204. Therefore, there is an electrical connection between the capacitive sensing module 254, the sensor processor module 252 and the processor module 116.

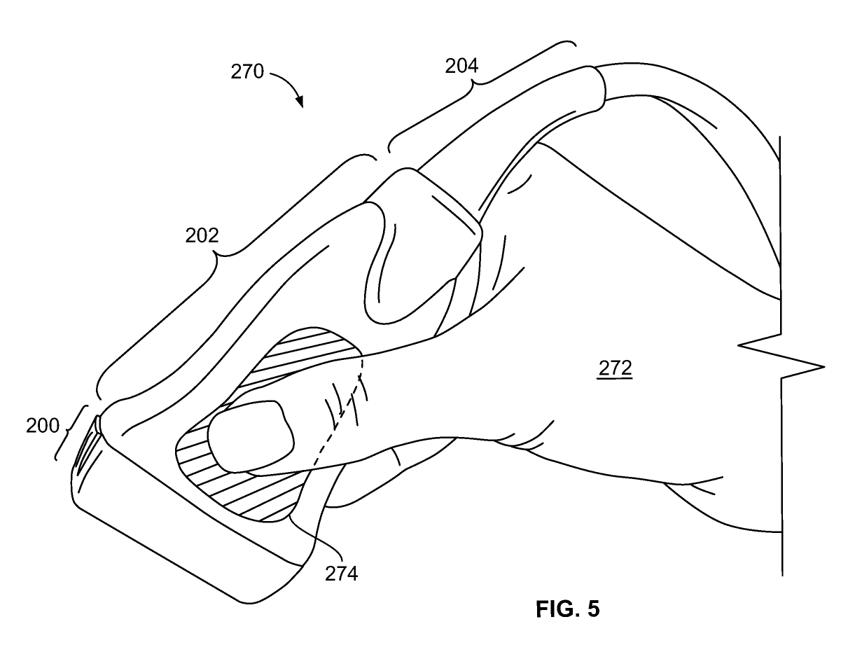

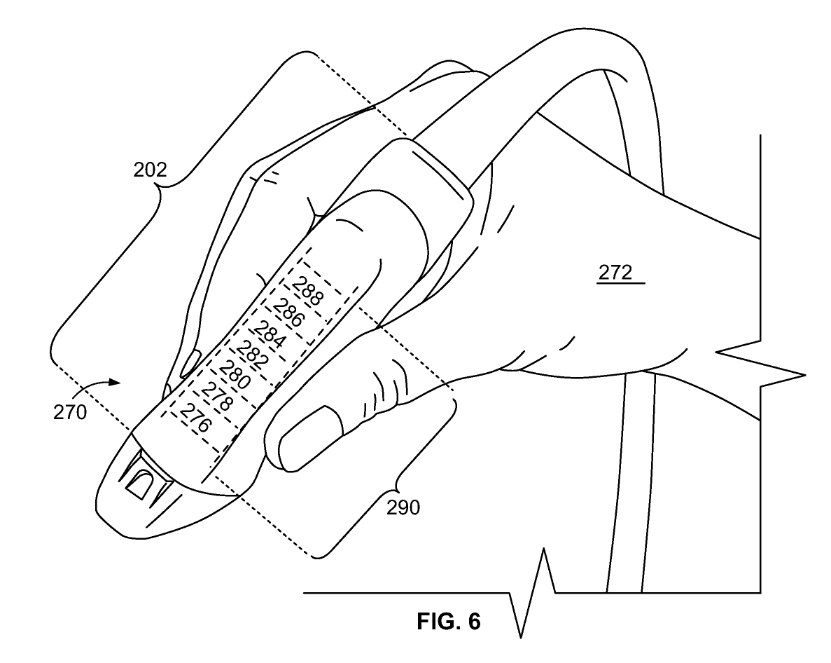

FIGS. 5 and 6 illustrate a touch sensitive probe 270 that has capacitive sensors incorporated within the housing 206 of the probe 270. In another embodiment, other touch sensing technology may be incorporated within the housing 206. The probe 270 is illustrated as being held by a user's hand 272 in a typical scanning position, wherein the user holds one side of the probe 270 with the thumb and the other side with one or more fingers. It should be understood that other shapes and sizes of probes are also contemplated and the embodiments discussed herein are not limited to any particular type of probe.

In FIG. 5, a plurality of capacitive sensors 240-250 (e.g. two or more capacitive sensors) may be incorporated within an area, such as area 274. Although not shown, a second area of capacitive sensors 240-250 may be formed on the other side of the probe 270. In one embodiment, one larger capacitive sensor 264 may be used to form the capacitive sensing layer 214 within the area 274. In another embodiment, the capacitive sensing layer 214 may extend over the entire handle 202 or most of the handle 202 of the probe 270, and the area 274 may be virtually mapped based on X, Y coordinates defining the outer surface 208. In yet another embodiment, the area 274 may be composed of an array of capacitive sensors that are implemented as an array of discrete sensors or overlapping sets of sensing elements forming a grid of sense points, similar to the sensor 264 of FIG. 4. The detection of contact with the area 274 may thus be virtually mapped based on X, Y coordinates defining the outer surface 208. When a grid of sense points is defined, the sensor processor module 252 may identify location(s) within the area 274 that are sensing or detecting a touch.

When the user picks up the probe 270, the level of capacitance to ground of one or more of the capacitive sensors 240-250 within the area 274 will increase. In one embodiment, when the level of capacitance is within a predetermined range or above a predetermined level, the system 100 senses that the probe 270 is being held by the user and may take an action, such as selecting or activating the probe 270. When the capacitance level is not within the predetermined range or above the predetermined level, the system 100 may sense that the probe 270 is not being held by the user and may take no action or may deactivate the probe 270 if the probe 270 is currently active. Therefore, it should be understood that contact between the user and the outer surface 208 of the probe 270 may be sensed and used to cause or initiate an action in the system 100. As discussed previously, a level of capacitance or other electrical characteristics or parameters may be sensed, such as resistance, inductance and/or pressure.

The capacitive sensors 240-250 within the area 274 may also detect levels of capacitance that result from changes in pressure. For example, an increase in the amount of force applied would result in more area of the deformable or compliant object (e.g. the finger) being in contact with the outer surface 208. The increased area of surface contact results in a higher level of capacitance that is associated with increased pressure or force. Therefore, the user may be able to squeeze or strobe the probe 270 to initiate an action, such as to advance a protocol (e.g. a series of discrete steps associated with an exam type or set-up operation) to a next step, save an image, print an image, and the like. In addition, the processor modules 116 and 252 may discriminate between signals received from the sensors 240-250 that indicate a constant hold and signals that indicate a tap, such as by tracking how long a capacitive sensor 240-250 outputs a certain level of capacitance.

Turning to FIG. 6, one or more virtual buttons 276, 278, 280, 282, 284, 286 and 288 may be formed within an area 290 by associating one or more capacitive sensors 240-250 (depending upon the size of the sensing area of each of the capacitive sensors 240-250) with each of the virtual buttons 276-288. Although not shown, additional virtual buttons may be provided on the opposite side of the handle 202 of the probe 270 or elsewhere along the outer surface 208 of the probe 270 and may be configured to be any size and shape. In one embodiment, if a larger sensor such as the sensor 264 is used to form the area 290 or to cover a portion or all of the outer surface 208 of the probe 270, the virtual buttons 276-288 may be mapped based on the X, Y coordinates of the sensor 264.

The term “virtual button” is intended to indicate a location defined on the probe 270 that is associated with or mapped to a particular function or action. Therefore, each of the virtual buttons 276-288 may be mapped to a different action, and the mapping may be based on, for example, a protocol that is running or active. For example, when the virtual button 276 is activated a first action may be taken and when the virtual button 278 is activated a second action may be taken that is different from the first action. When a different protocol is active, the virtual buttons 276 and 278 may be associated with two actions that are different from the first and second actions. An indication (not shown) may be formed or printed on the outer surface 208 to identify the locations of each of the virtual buttons 276-288.

The virtual buttons 276-288 may be selected or activated when a touch is sensed or when an increase in pressure results in a further increase in the level of capacitance. In another embodiment, the processor module 116 or 252 may be configured to detect when one or more of the virtual buttons 276-288 are experiencing a constant hold. Therefore, if the user were holding the probe 270 in a manner in which a part of the hand was in contact with at least one of the virtual buttons 276-288, the virtual buttons 276-288 would not be erroneously activated.

By way of example, the user interface 124 may be used to map the capacitive sensors 240-250 and 264 incorporated in the probe 270, such as by viewing a diagram of the probe 270, which in some embodiments may include X, Y location information, or a list on the display 118. This may enable the user to map more than one virtual button or area within a large sensor 264. Some capacitive sensors 240-250 may not be mapped to an action, and thus any change in capacitance may be ignored. For example, the user may configure the same types of probes to operate in the same way for each system at a site, or may configure the probes based on individual users of the system. In another embodiment, a default set of behaviors may be programmed based on probe or system type.

Each of the virtual buttons 276-288 may be programmable based on user preference. Therefore, a particular site or user may program each of the probes 270 to respond in the same manner, facilitating the ease of use between different ultrasound systems 100. By way of example only, the virtual buttons 276-288 may be used to change or select an imaging mode, such as from within B-mode, M-mode, Doppler and color flow modes, or any other mode available to the system 100 or the probe 270. The virtual buttons 276-288 may also be used to move or scroll through menus, lists and the like to make selections, capture images, optimize image parameters, make changes to the display 1 18, annotate, or any other action that is selectable from the user interface 124.

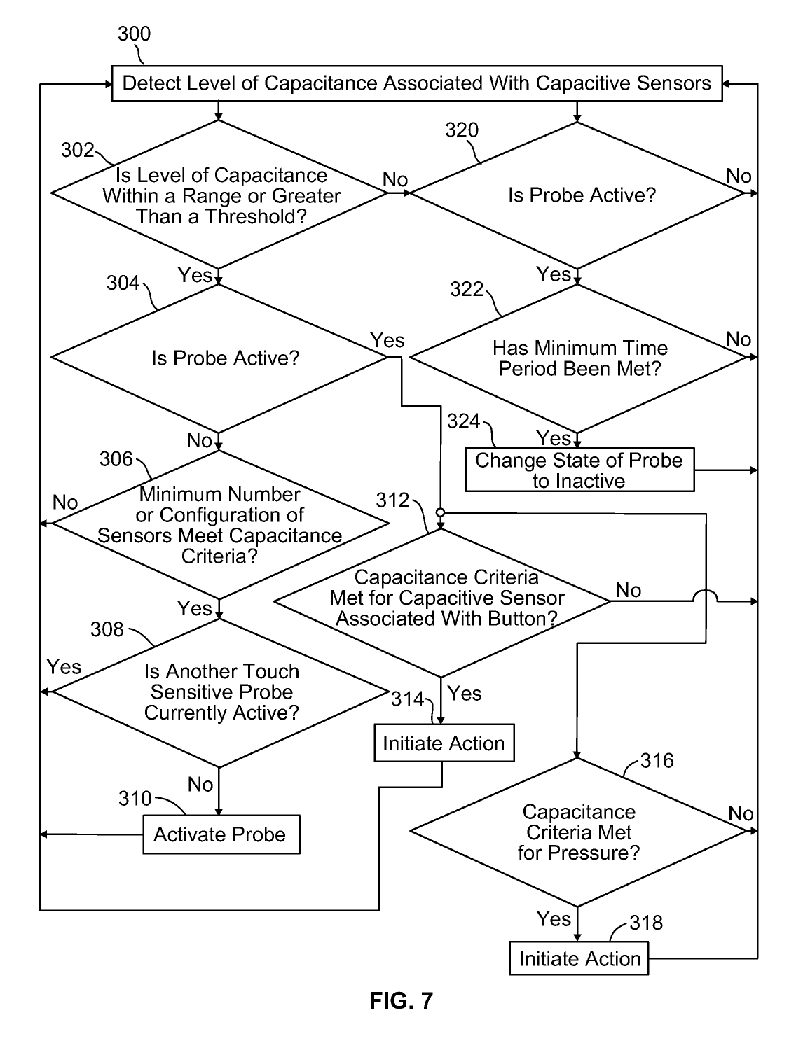

FIG. 7 illustrates a method for using the probe 106 or 270 that has at least one sensor capable of detecting a touch, such as at least one capacitive sensor 240-250, integrated into the housing 206. When using the probe 106 or 270 that has touch sensing functionality within the housing 206, the number of user inputs and/or movements, such as entering selections through the user interface 124 during an exam, may be reduced. In one embodiment, the system 100 may provide a minimum level of power to each probe 270 that is connected to the system 100 to power the sensor processor module 252 and/or capacitive sensors 240-250. The method of FIG. 7 is primarily discussed with respect to capacitive sense technology. However, it should be understood that other touch sensing technologies may similarly be used.

At 300, the capacitive sensing module 254 senses or detects a level of capacitance associated with each of the capacitive sensors 240-250. In another embodiment, a sensing module may detect a level of a different electrical parameter, such as resistance or inductance. It should be understood that if more than one touch sensitive probe is connected to the system 100, there would be multiple capacitive sensing modules 254 detecting capacitance levels associated with the different probes. Therefore, multiple touch sensitive probes may be monitored at the same time. Also, each of the touch sensitive probes are sensed as soon as the probe is connected to the probe port 120.

The capacitive sensing module 254 and/or the sensor processor module 252 or 116 may determine, at 302, whether any of the capacitive sensors 240-250 have a level of capacitance that satisfies a capacitance criteria, such as being within a predetermined range or being greater than a predetermined level or threshold. In one embodiment, the predetermined range may be approximately 0.1 picofarad (pf) to fifty pf. In another embodiment, the predetermined level may be approximately one pf. However, it should be understood that other ranges and levels may be used. For example, different capacitive sensor geometries, manufacturers and/or manufacturing processes may set different ranges and/or levels that correspond to the detection of a human or organic touch on the outer surface 208. Therefore, if the system 100 detects that the level of capacitance is less than the predetermined level or threshold, such as less than 0.1 pf or one pf, the system 100 may associated that level of capacitance with a probe holder or table, for example. Similarly, if other types of sensors are used, other ranges and levels or thresholds may be determined based on the particular parameter(s) being detected.

If one or more capacitive sensors 240-250 meet the capacitance criteria, the method passes to 304 where the sensor processor module 252 determines whether the probe 270 is active. If the probe 270 is not active, the method passes to 306. At 306, in some embodiments the sensor processor module 252 may determine whether a minimum number of the capacitive sensors 240-250 or a predetermined configuration of the capacitive sensors 240-250 have capacitance values that fall within the predetermined range or are above the threshold. For example, the sensor processor module 252 may ignore the capacitance changes unless at least two (or some other minimum number) of capacitive sensors 240-250 meet the capacitance criteria. In another embodiment, the sensor processor module 252 may ignore the capacitance changes unless at least one capacitive sensor 240-250 located on each of the opposite sides of the probe 270 meet the capacitance criteria. For example, the sensor processor module 252 may ignore the capacitance changes unless at least one capacitive sensor 240-250 within the area 274 (as shown in FIG. 5) and at least one capacitive sensor 240-250 within the area on the opposite side of the probe 270 meet the criteria, indicating that the probe 270 is being held by the hand of the user. This may prevent the processor module 116 from accomplishing an action based on an erroneous touch.

In one embodiment, the sensor processor module 252 may ignore the capacitance changes unless the capacitive sensors 240-250 have maintained a level of capacitance for a minimum period of time, such as one second or two seconds. The processor module 116 or 252 may not initiate any action until the period of time has passed.

In another embodiment, when the probe 270 is not active the capacitive sensing module 254 may ignore any change in a portion of the capacitive sensors, such as the capacitive sensors associated with the virtual buttons 276-288. In other words, some of the capacitive sensors may have functionality that is only recognized when the probe 270 is actively being held by the user. In yet another embodiment, if a minimum number of the capacitive sensors associated with the virtual buttons 276-288 are sensed as having a constant hold while the probe 270 is not active, the sensor processor module 252 may determine that the user is holding the probe 270 on that side and thus not ignore the capacitive changes.

If the minimum number or configuration of capacitive sensors 240-250 do not have capacitance values that are within the capacitance criteria, the method returns to 300. If the capacitance criteria are met at 306, the sensor processor module 252 may communicate a selection signal or other identifying information to the processor module 116 for identifying which of the capacitive sensors 240-250 meet the capacitance criteria. The method passes to 308 where the processor module 116 determines whether another touch sensitive probe is currently active. If no, the method passes to 310 where the processor module 116 may activate the probe 270 and put the system 100 and the probe 270 into a predetermined state, such as a scanning or imaging state. This may eliminate two or more selections the user would typically make through the user interface 124. In another embodiment, the processor module 116 may select the probe 270 without placing the probe 270 in an imaging state. In yet another embodiment, the processor module 116 may activate a particular protocol associated with the probe 270 in addition to or instead of activating the probe 270.

If at 308 another touch sensitive probe is currently active, the method may return to 300 and the currently detected probe 270 is not activated. For example, a user may be adding or connecting a touch sensitive probe to the system 100 and thus may not want the new probe to be activated. In another embodiment, if at 308 a probe that is not touch sensitive is already active, user defined criteria may be used to determine which probe should be active. For example, if a probe that is not touch sensitive is active, the processor module 116 may ignore touch information sensed from any touch sensitive probe. In another embodiment, touch sensitive probes may be defined as having a higher priority and may thus be activated while the probe that is not touch sensitive may be deactivated.

Returning to 304, if the probe 270 is active the method passes to 312 and 316. At 312, if the capacitive sensing module 254 senses a capacitance level within the predetermined range that is associated with one of the capacitive sensors that form the virtual buttons 276-288, the method passes to 314 where the processor module 116 will initiate the associated action. In one embodiment, the sensor processor module 252 may output a corresponding selection signal to the processor module 116 via wires 256. As discussed previously, each of the virtual buttons 276-288 may be associated with a particular protocol, action, action within a protocol, scan setting, screen display and the like.

At 316, when the probe 270 is active the sensor processor module 252 may compare the level of capacitance to a higher threshold that indicates that the user has applied force or pressed on the capacitive sensor 240-250. For example, the primary sensing area 274 may be strobed by the user by loosening and tightening a grip. If the sensor processor module 252 detects that the capacitance criteria has been met for pressure, the method passes to 318 where the processor module 116 initiates a predetermined action. For example, the processor module 116 may respond to the short time duration of increased pressure, reflected by an increase in capacitance, by advancing the currently active protocol to the next step. Therefore, the user may utilize a touch, light tap or slight increase in pressure on the outer surface 208 of the probe 270 to advance the protocol to a next step, step through options, make a selection, or otherwise initiate an action. This reduces the number of times the user has to interact with the user interface 124 and may increase the user's efficiency.

Returning to 302, if no capacitive sensors 240-250 meet the capacitance criteria, the method passes to 320 where the processor module 116 may determine whether the probe 270 is active. If the probe 270 is not active, the method returns to 300. If the probe 270 is active, the processor module 116 may determine at 322 whether a minimum time period, such as one or two seconds, has passed since the capacitance criteria has been met. This time period may allow the user to change a grip on the probe 270 without changing the currently selected operation, probe activation, protocol and the like. If the mini mum time period has been met, at 324 the processor module 116 may change the state of the probe 270 to inactive. Therefore, the probe 270 is no longer consuming power. The method then returns to 300.

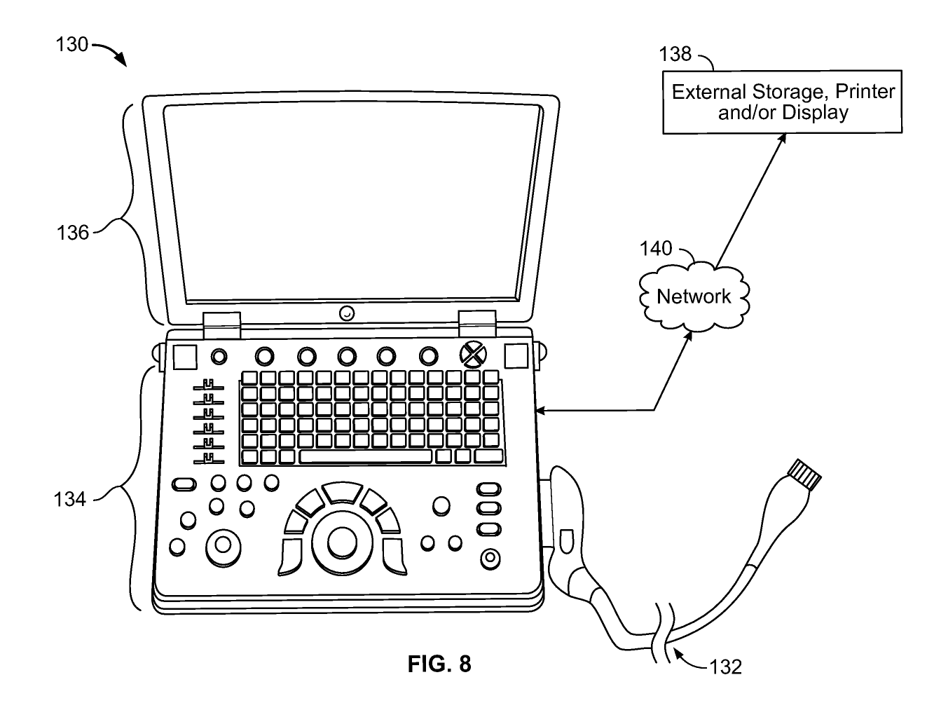

FIG. 8 illustrates a 3D-capable miniaturized ultrasound system 130 having a probe 132 that has touch sensing technology, such as at least one capacitive sensor 240-250, incorporated within the housing of the probe 132. The probe 132 may be configured to acquire 3D ultrasonic data. For example, the probe 132 may have a 2D array of transducer elements 104 as discussed previously with respect to the probe 106 of FIG. 1. A user interface 134 (that may also include an integrated display 136) is provided to receive commands from an operator in addition to the input sensed through the capacitive sensors 240-250. As used herein, “miniaturized” means that the ultrasound system 130 is a handheld or hand-carried device or is configured to be carried in a person's hand, pocket, briefcase-sized case, or backpack. For example, the ultrasound system 130 may be a hand-carried device having a size of a typical laptop computer, for instance, having dimensions of approximately 2.5 inches in depth, approximately 14 inches in width, and approximately 12 inches in height. The ultrasound system 130 may weigh about ten pounds, and thus is easily portable by the operator. The integrated display 136 (e.g., an internal display) is also provided and is configured to display a medical image.

The ultrasonic data may be sent to an external device 138 via a wired or wireless network 140 (or direct connection, for example, via a serial or parallel cable or USB port). In some embodiments, external device 138 may be a computer or a workstation having a display. Alternatively, external device 138 may be a separate external display or a printer capable of receiving image data from the hand carried ultrasound system 130 and of displaying or printing images that may have greater resolution than the integrated display 136. It should be noted that the various embodiments may be implemented in connection with a miniaturized ultrasound system having different dimensions, weights, and power consumption.

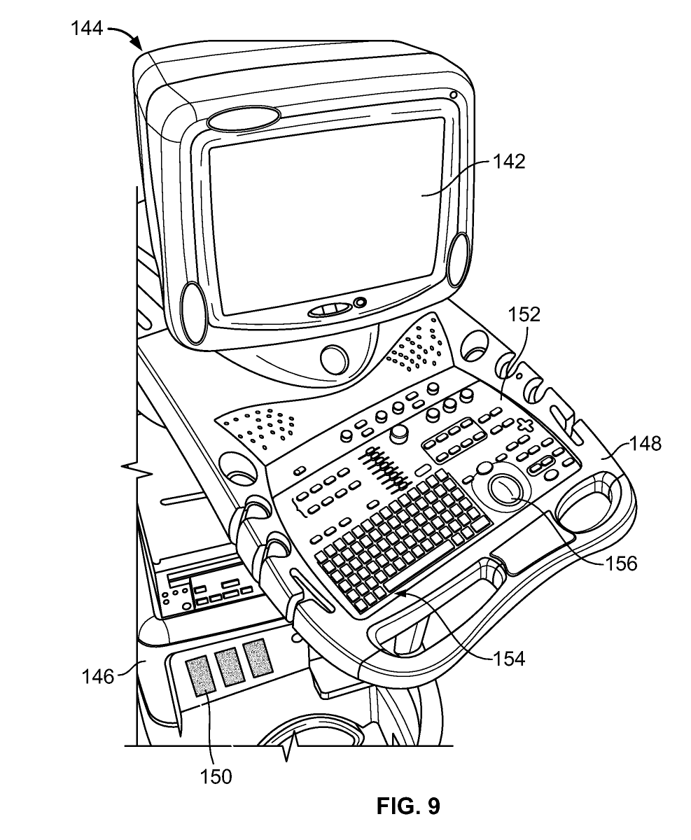

FIG. 9 illustrates a mobile ultrasound imaging system 144 provided on a movable base 146. The ultrasound imaging system 144 may also be referred to as a cart-based system. A display 142 and user interface 148 are provided and it should be understood that the display 142 may be separate or separable from the user interface 148.

The system 144 has at least one probe port 150 for accepting probes, such as the probe 106 and 270 that have touch sensing functionality integrated there-within. Therefore, the user may control various functions of the system 144 by touching or pressing on the outer surface 208 of the probe 270.

The user interface 148 may optionally be a touchscreen, allowing the operator to select options by touching displayed graphics, icons, and the like. The user interface 148 also includes control buttons 152 that may be used to control the ultrasound imaging system 144 as desired or needed, and/or as typically provided. The user interface 148 provides multiple interface options that the user may physically manipulate to interact with ultrasound data and other data that may be displayed, as well as to input information and set and change scanning parameters. The interface options may be used for specific inputs, programmable inputs, contextual inputs, and the like. For example, a keyboard 154 and track ball 156 may be provided.

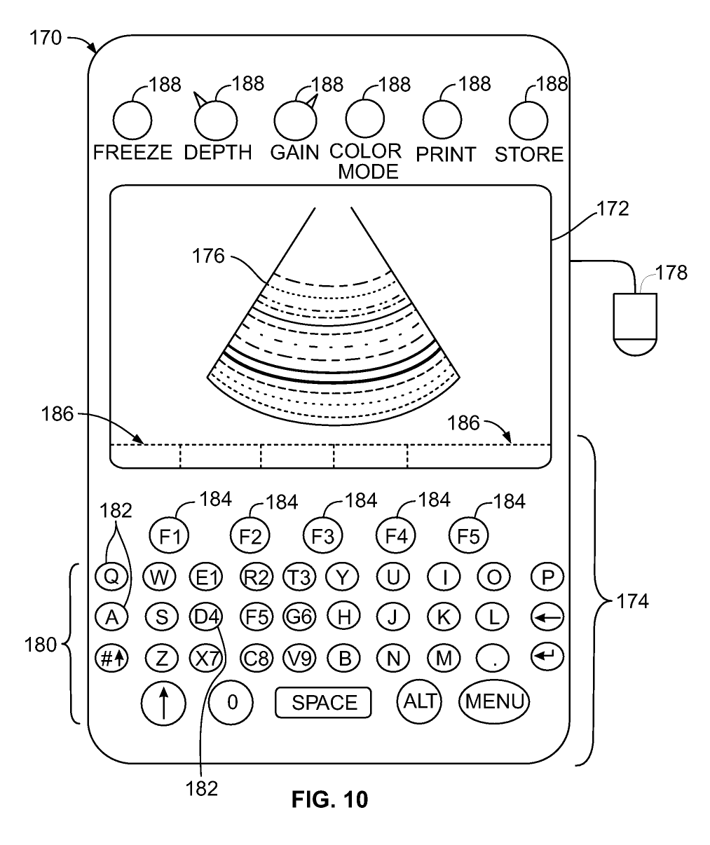

FIG. 10 illustrates a hand carried or pocket-sized ultrasound imaging system 170 wherein display 172 and user interface 174 form a single unit. By way of example, the pocket-sized ultrasound imaging system 170 may be approximately 2 inches wide, approximately 4 inches in length, and approximately 0.5 inches in depth and weighs less than 3 ounces. The display 172 may be, for example, a 320×320 pixel color LCD display (on which a medical image 176 may be displayed). A typewriter-like keyboard 180 of buttons 182 may optionally be included in the user interface 174. A touch sensing probe 178 having one or more sensors integrated within the housing to detect touch on an outer surface of the probe 178 is interconnected with the system 170. Therefore, whenever the user is not holding the probe 178, the probe 178 may be inactive or in a battery-extending low-power mode.

Multi-function controls 184 may each be assigned functions in accordance with the mode of system operation. Therefore, each of the multi-function controls 184 may be configured to provide a plurality of different actions. Label display areas 186 associated with the multi-function controls 184 may be included as necessary on the display 172. The system 170 may also have additional keys and/or controls 188 for special purpose functions, which may include, but are not limited to “freeze,” “depth control,” “gain control,” “color-mode,” “print,” and “store.”

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the fill scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An ultrasound probe, comprising:
a probe housing comprising an inner surface and an outer surface;
an array of transducer elements within the probe housing; and
at least one sensor formed between the inner and outer surfaces of the probe housing, the at least one sensor configured to detect at least one parameter associated with an object in contact with the outer surface proximate the at least one sensor.

2. The probe of claim 1, wherein the probe housing comprises a layer of plastic formed proximate the outer surface, and wherein the at least one sensor is positioned proximate the inner surface.

3. The probe of claim 1, wherein the at least one sensor is integrated within the probe housing.

4. The probe of claim 1, wherein the at least one sensor comprises a plurality of capacitive sensors configured to provide capacitive sensing within at least one predetermined area of the outer surface of the probe.

5. The probe of claim 1, further comprising a sensor processor module configured to generate a selection signal associated with an action when a detected level of the at least one parameter is one of within a predetermined range and at a desired relationship with respect to a predetermined threshold.

6. The probe of claim 1, wherein the at least one sensor comprises at least one of a capacitive sensor, an inductive sensor, a resistance sensor, and a piezoelectric element.

7. The probe of claim 1, wherein the at least one sensor is a non-mechanical sensor.

8. The probe of claim 1, wherein a detected level of the at least one parameter is used to determine a type of action to be performed.

9. An ultrasound system, comprising:
an ultrasound probe comprising:
a probe housing comprising an inner surface and an outer surface;
an array of transducer elements within the probe housing; and
at least one sensor formed between the inner and outer surfaces of the probe housing, the at least one sensor configured to detect a level of at least one parameter associated with an object in contact with the outer surface proximate the at least one sensor; and
a processor module electrically coupled to the ultrasound probe, the processor module configured to initiate an action based on a relationship of the level of the at least one parameter to predetermined criteria.

10. The system of claim 9, wherein the processor module is further configured to one of activate and select the probe when the probe is not active and the level of the at least one parameter satisfies the criteria.

11. The system of claim 9, wherein the processor module is further configured to deactivate the probe when the probe is active and the level of the at least one parameter is outside the criteria.

12. The system of claim 9, wherein the at least one parameter is at least one of capacitance, resistance, inductance, pressure and voltage.

13. The system of claim 9, wherein the at least one parameter is capacitance, and wherein the processor module is further configured to initiate an action when the level of capacitance is greater than a second threshold that corresponds to an increase in pressure associated with the object in contact with the outer surface.

14. The system of claim 9, wherein the at least one sensor is configured to provide a plurality of virtual buttons associated with areas on the outer surface of the probe housing that are each associated with a different action, and wherein the processor module is further configured to initiate the associated action when the area of the outer surface corresponding to the virtual button is in contact with the object.

15. A method for controlling an ultrasound system based on capacitance changes detected proximate to an outer surface of an ultrasound probe, the method comprising:
detecting with at least one capacitive sensor a level of capacitance on an outer surface of an ultrasound probe;
comparing the level of capacitance to a capacitance criteria with a processor module; and
initiating an action with the processor module when the level of capacitance satisfies the capacitance criteria.

16. The method of claim 15, further comprising:
determining that the probe is inactive; and
automatically activating the probe when the level of capacitance satisfies the capacitance criteria.

17. The method of claim 15, further comprising:
determining that the probe is active; and
automatically deactivating the probe when the level of capacitance is outside the capacitance criteria for a predetermined period of time.

18. The method of claim 15, wherein the at least one capacitive sensor comprises a plurality of capacitive sensors, wherein at least one of the capacitive sensors is associated with a first action and a different one of the capacitive sensors is associated with a second action, the first and second actions being different with respect to each other.

19. The method of claim 15, wherein the action is determined based on at least one of a status of the probe, a probe type, a protocol, user preset parameters and site preset parameters.

20. The method of claim 15, wherein the action is one of activating the probe, deactivating the probe, selecting a protocol, advancing a protocol, and selecting an option.
